(19) United States
(12) Plant Patent Application Publication
(10) Pub. No.: US 2006/0026725 P1
(43) Pub. Date: Feb. 2, 2006

(54) Pear tree named 'Crispie'

(50) PYRUS COMMUNIS X PYRUS PYRIFOLIA; PEAR

(76) Inventor: Allan G. White, Havelock North (NZ)

Correspondence Address: GREENLEE WINNER AND SULLIVAN P C, 4875 PEARL EAST CIRCLE, SUITE 200, BOULDER, CO, 80301, US

(21) Appl. No.: 10/900,689

(22) Filed: Jul. 27, 2004

(51) Int. Cl. A01H 5/00

(52) U.S. Cl. PLT/176

(57) ABSTRACT

A new distinct pear variety is described. This interspecific variety results from crossing between ‘Nijisseiki’ (Pyrus pyrifolia Nakai) (not patented) and ‘Max Red Bartlett’ (Pyrus communis) (U.S. Plant Pat. No. 741). The new variety has been named ‘Crispie’ and was selected because of its crisp texture and ‘Bartlett’ type flavour. The fruit of this new variety is characterised by its pyriform shape, smooth yellow skin, crisp texture and ‘Bartlett’ type flavour when fully ripe.

### GENUS AND SPECIES OF PLANT CLAIMED

Pyrus communis×Pyrus pyrifolia

### BACKGROUND OF THE INVENTION

A new distinct pear variety is described. This interspecific variety results from crossing between ‘Nijisseiki’ (Pyrus pyrifolia Nakai) (not patented) and ‘Max Red Bartlett’ (Pyrus communis) (U.S. Plant Pat. No. 741). The new variety has been named ‘Crispie’ and was selected because of its crisp texture and ‘Bartlett’ type flavour. The fruit of this new variety is characterised by its pyriform shape, smooth yellow skin, crisp texture and ‘Bartlett’ type flavour when fully ripe.

### SUMMARY OF THE INVENTION

This new variety was selected from a population of seedlings derived from crossing the pear varieties ‘Nijisseiki’ and ‘Max Red Bartlett’. The cross was made in 1989 at Hawkes Bay, New Zealand. The variety was determined to be distinct from the parent varieties by the following characteristics:
Nijisseiki: ‘Crispie’ has strong tree vigour compared to medium vigour of ‘Nijisseiki’, pyriform fruit shape compared to the round shape of ‘Nijisseiki’, ‘Bartlett’ like flavour when fully ripe compared to a mild subtle flavour of ‘Nijisseiki’.
Max Red Bartlett: The tree of ‘Crispie’ is vigorous compared to medium vigour of ‘Max Red Bartlett’. ‘Crispie’ fruits on spurs and heavily on one year old wood while ‘Max Red Bartlett’ fruits mostly on spurs. ‘Crispie’ fruit has yellow skin colour and crisp texture compared to the red skin colour of ‘Max Red Bartlett’ and soft texture.
Williams' Bon Chretien: The tree of ‘Crispie’ is vigorous compared to medium vigour of ‘Williams' Bon Chretien’. ‘Crispie’ fruits on spurs and heavily on one year old wood while ‘Williams' Bon Chretien’ fruits mosty on spurs. ‘Crispie’ fruit has a narrow calyx end shape when compared with ‘Williams' Bon Chretien’. ‘Crispie’ has crisp texture compared to the soft texture of ‘Williams' Bon Chretien’.

The new variety has been named ‘Crispie’. True to type budding and grafting onto Quince rootstock ‘BA29’ with ‘Buerre Hardy’ pear as an interstock has been used to obtain plants. Asexual propagation by budding and grafting has shown that the unique combinations of characteristics are consistently transferred through successive propagation.

### BRIEF DESCRIPTION OF THE DRAWINGS

The accompanying photographs show typical specimens of the tree, foliage, fruit of the new variety as depicted in colours nearly as true as is reasonably possible to make the same colour illustration of this character.

FIG. 1: side view of typical fruit of the pear variety ‘Crispie’.

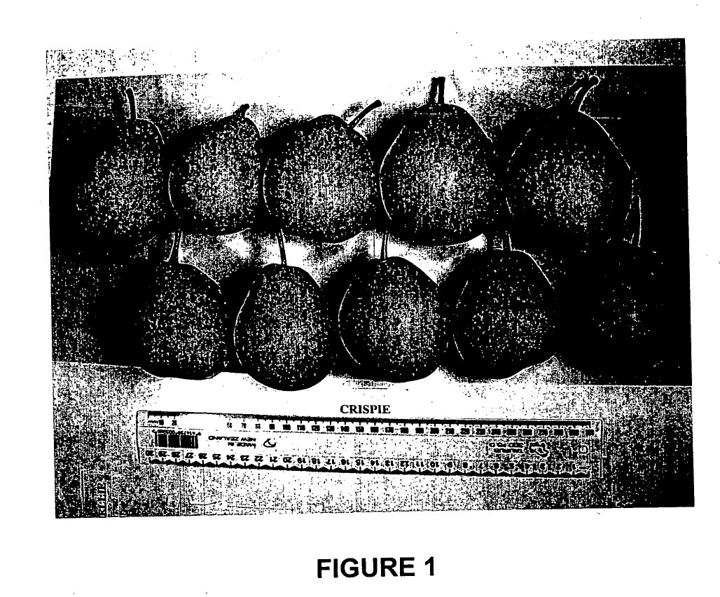

FIG. 2: calyx end view of typical fruit of the pear variety ‘Crispie’.

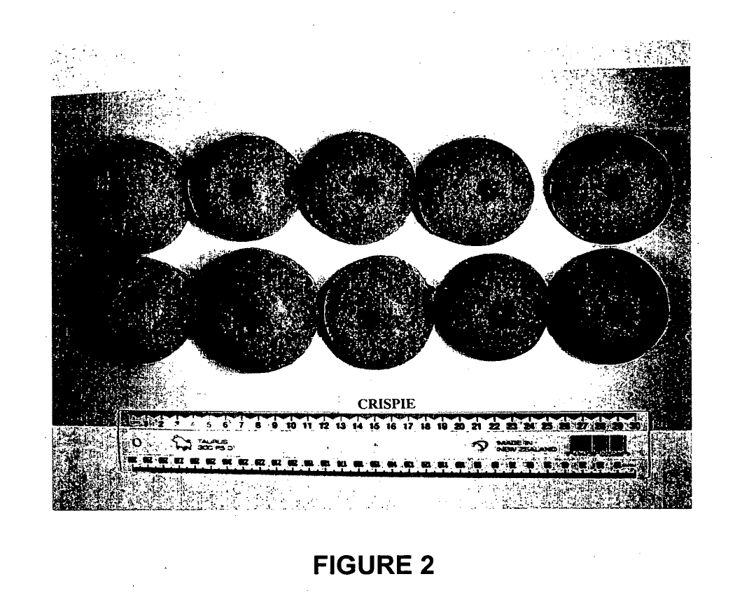

FIG. 3: stem end view of a typical fruit of the pear variety ‘Crispie’.

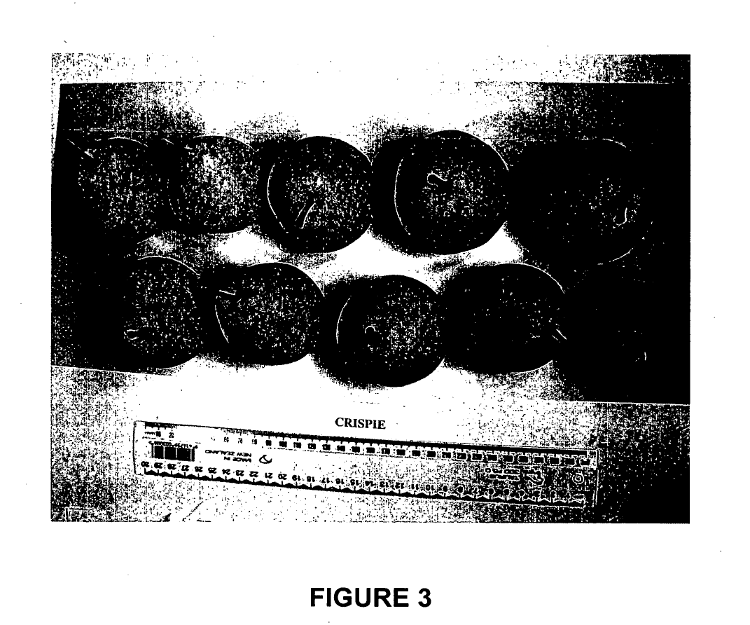

FIG. 4: cross sectional view of a typical fruit of the pear variety ‘Crispie’.

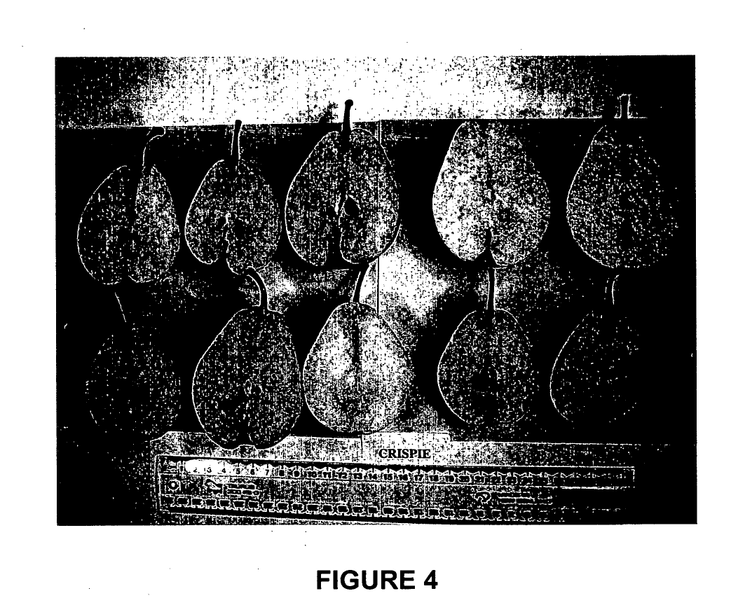

FIG. 5: typical mature leaves of ‘Crispie’ showing the upper and lower surfaces.

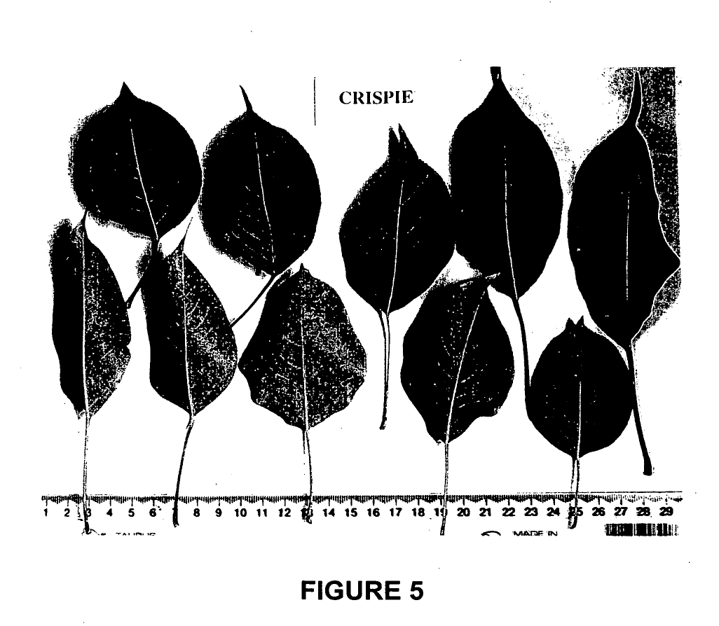

FIG. 6: cross sectional fruit view of ‘Nijisseiki’, (far left), ‘Crispie’ (middle), ‘Williams' Bon Chretien’ (right).

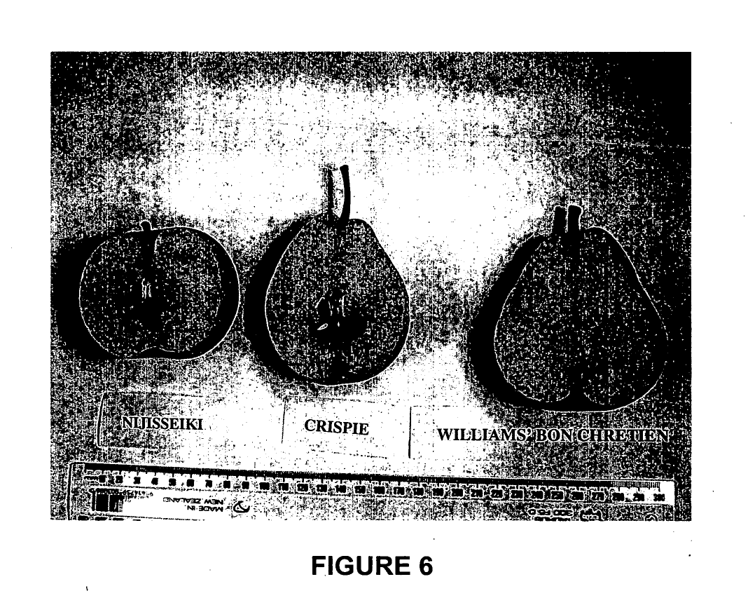

FIG. 7: fruiting spur ‘Crispie’.

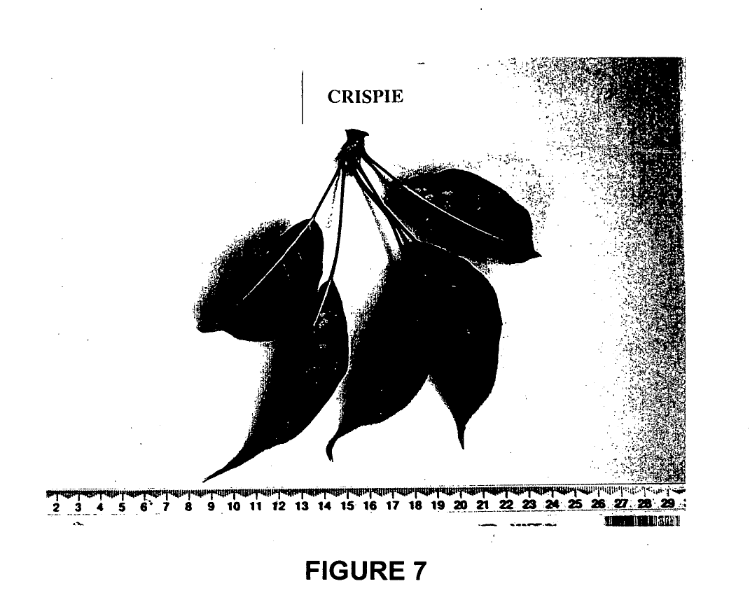

FIG. 8: tree of variety ‘Crispie’.

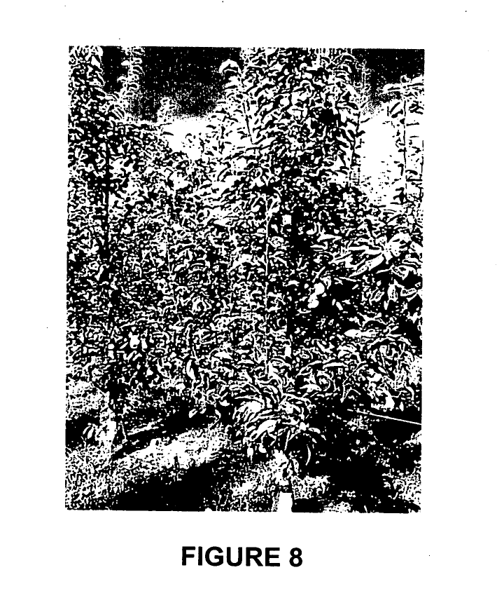

### MORPHOLOGICAL DESCRIPTION OF THE VARIETY

The following is a detailed description of the new variety with colour terminology in accordance with The Royal Horticultural Society Colour Charts (R.H.S.C.C.) 2001 edition.

Observations were made in the 2003-2004 season on trees grafted onto Quince ‘BA29’ rootstocks with a ‘Buerre Hardy’ pear interstock, planted in 1999.
Tree: Vigour strong when grafted onto Quince ‘BA29’ rootstocks with a ‘Buerre Hardy’ interstock; tree size large; branching strong; habit spreading; bearing on spurs and 1 year wood.
Trunk: Bark grey R.H.S.C.C. N200D, when mature; diameter of 218 mm at a height of 200 mm.
Branches: Strong; number high; approximately 3 spurs/100 mm of 2 year old wood; high annual growth numbers with a length of 400-500 mm; annual growth wood is greyed orange R.H.S.C.C. 166A.
Lenticels: Numbers on current years growth medium; size large.
Leaves: Average length 103.5 mm; width 60.2 mm; average length/width ratio medium; upward pose; shape in cross section up folded; indentations of the margin sharply serrate, medium depth; shape of leaf blade apex (excluding pointed tip) right angled; petiole average length 38.1 mm; stipules absent; colour of leaf blade green R.H.S.C.C. 146A.
Flower buds: flower bud length medium.
Flowers: Bloom period medium late, from 24^{th} September to 15^{th} October in Nelson, New Zealand, full bloom date 4^{th} October; sepal length medium; sepal attitude in relation to the corolla recurved; margins of petals overlapping; position of stigma in relation to stamens same level; size of petal medium; shape of petal (excluding claw) circular; shape of petal base (excluding claw) cuneate; length of claw petal short.
Fruit: Fruit is mature for consumption by the third week of February; harvest period is from 10 February to 20 February, in Nelson, New Zealand.
Size: medium large; average fruit weight 214.8 g; average width 71.1 mm, average height 85.6 mm; fruit length/diameter ratio medium; maximum diameter slightly towards calyx of fruit.
Shape: pyriform; slightly asymmetric in longitudinal section; profile of the sides straight.
Eye basin: Average width 24.5 mm; average depth 4.9 mm, relative area of russet around eye basin very small.
Stalk: Thickness of the stalk medium, medium length 21.7 mm, curvature of stalk weak to medium, the attitude of the stalk is oblique in relation to the axis of the fruit; depth of stalk cavity shallow average 2.6 mm, amount of russet around the stem cavity small.
Sepal: Calyx or sepals are partially persistent, approximately 10% of fruit at harvest have a deciduous calyx and sepals, remaining 90% have upright sepals.
Skin: smooth; greasiness absent, cracking tendency of skin absent; background colour yellow R.H.S.C.C. 8B; lenticels small.
Overcolour: Absent.
Texture: Crisp; flesh medium; firmness medium; flesh juiciness medium.
Flavour: Sweetness medium; acidity low; ‘Bartlett’ like aroma when ripe.
Quality: Very good.
Seeds: Five locules, 1 to 2 seeds per locule; medium seed size 11 mm long, 5 mm wide, colour black R.H.S.C.C. 202A.
Use: Dessert.
Production: Early season, regular cropping when thinned.
Management: Thinning of crop load in early summer is recommended. Early cropping is required to keep vegetative growth in balance.
Pest and disease: Susceptible to pear slug (Caliroa cerasi). Highly leaf and fruit resistant to pear scab (Venturia pirina Adherh).

I claim:

1. A new and distinct variety of pear tree named ‘Crispie’ substantially as herein illustrated and described.
